(19) United States
(12) Patent Application Publication
(10) Pub. No.: US 2026/0073513 A1
(43) Pub. Date: Mar. 12, 2026

(54) BODY IMAGING AND DIAGNOSTIC SYSTEM

(71) Applicant: oVio Technologies, Inc., Newport Beach, CA (US)

(72) Inventor: George Rebensdorf, Newport Beach, CA (US)

(21) Appl. No.: 19/262,598

(22) Filed: Jul. 8, 2025

Related U.S. Application Data
(60) Provisional application No. 63/668,514, filed on Jul. 8, 2024.

(51) Int. Cl. G06T 7/00; G06T 7/11; G16H 50/20; G16H 50/70

(52) U.S. Cl. CPC G06T 7/0012; G06T 7/11; G16H 50/20; G16H 50/70; G06T 2207/10052; G06T 2207/20081; G06T 2207/20084; G06T 2207/30088

(57) ABSTRACT

A method of detecting skin conditions includes capturing a first image scan, identifying areas of interest of the first image scan using a trained custom image segmentation model, classifying each of the areas of interest to correspond to skin conditions, routing each of the areas of interest to at least one trained specialist model, classifying the areas of interest using the at least one trained specialist model, extracting a deep feature layer of the at least one trained specialist model for each of the areas of interest, calculating a skin condition metric for each of the areas of interest using the deep feature layer, displaying the skin condition metric associated with each of the areas of interest, and displaying ranked areas of interest identified in the first image scan.

### CROSS-REFERENCE TO RELATED APPLICATIONS

This application claims the benefit of U.S. Provisional Application No. 63/668,514, filed on Jul. 8, 2024, the entirety of which is incorporated by reference herein.

### FIELD OF THE INVENTION

The present invention relates to imaging and diagnostics systems, and more particularly to a 360-degree imaging system that analyzes images and provides diagnostic information to detect body changes; scars, marks, tattoos, and lesions; and skin abnormalities or conditions.

### BACKGROUND OF THE INVENTION

In the field of medicine and other fields such as forensics, professionals often need to identify physiological changes on a subject's body, including to diagnose or determine an identity of the subject. It also may be advantageous to diagnose or determine skin abnormalities or conditions on a subject's body. In addition, skin abnormalities or conditions are increasingly common and require significant time and expense to diagnose and treat them. For example, the increasing incidence of skin cancer, particularly melanoma, which has risen by 46% over the past 15 years, highlights the critical need for accurate and early detection. A naked eye observation and other current imaging techniques are insufficient in providing an efficient and thorough analysis. Traditional methods of lesion evaluation, such as dermoscopy and near-field imaging, present several limitations. Dermoscopy is generally time-consuming and heavily dependent on the clinician's experience and expertise. Near-field imaging often lacks the broader context necessary for accurate diagnosis and can result in significant numbers of false-positives. In addition, single-image format artificial intelligence (AI) systems tend to lack context for both inter-patient and intra-patient trends that might be included in a traditional clinician approach. Still further, primary care physicians may not have tools or systems that can adequately assist them in referring to specialists in fields such as dermatology, or the like. Accordingly, a need exists for methods and systems that provide consistent images of patients while achieving a high level of efficiency and accuracy to assist a clinician in identifying areas of interest or suspicious skin abnormalities.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a method of detecting skin abnormalities. In accordance with the method, a first image scan is captured, areas of interest of the first image scan are identified using a trained image segmentation model, wherein the trained image segmentation model is trained using a first plurality of image scans, the areas of interest are classified in accordance with classifications of a trained deep neural network trained with a second plurality of image scans, wherein the classifications include at least one of suspicious pigmented lesions (SPLs), medium-priority nonsuspicious pigmented lesions (NSPLs), and low-priority NSPLs, a deep feature layer of the trained neural network is extracted for each of the areas of interest, an ugly duckling metric is calculated for each of the areas of interest by comparing a geometric distance of the deep feature layer with an average of the deep feature layer for the areas of interest, the ugly duckling metric associated with each of the areas of interest is displayed in accordance with the image scan, and ranked areas of interest identified in the first image scan are displayed, ranked in accordance with the classifications. Each of the first image scan and the first and second pluralities of image scans may include a 360-degree view of at least a portion of a body part.

The first image scan may be stored in a database, wherein the database comprises a dataset of images of body parts including the first image scan. The dataset of images may include the first and second pluralities of image scans. The areas of interest may include at least one of lesions, burns, moles, freckles, tan lines, wrinkles, cuts, bruises, scars, marks, tattoos, bumps, and lumps. The first image scan may include 360 individual images with consistent lighting, position, and backdrop.

The first image scan may be captured with a 360-degree imaging device. The 360-degree imaging device may include a rotating unit that includes an imaging camera, wherein the rotating unit is rotatable between a home position and a finish position about a rotation axis such that the imaging camera is configured to capture the first scan, an alignment camera configured to capture a first alignment image of a subject positioned generally co-axially with the rotation axis, and a first display on which the first alignment image is displayed.

The first image scan also may be captured with a 360-degree imaging device that includes a rotating unit that includes an imaging camera. The rotating unit is rotatable about a rotation axis, wherein the rotating unit includes a first portion and a second portion that are rotatable about the rotation axis. The first portion and the second portion each have a proximal end through which the rotation axis extends and a distal end that is positioned away from the rotation axis. The imaging camera is associated with the distal end of the first portion and a backdrop is associated with the distal end of the second portion. The first portion and the second portion are rotatable from a home position where the distal ends of the first portion and the second portion are approximately 180° from one another with respect to the rotation axis. The first portion and the second portion are rotatable together between the home position and a finish position such that the imaging camera can capture the first scan of a subject positioned generally co-axially with the rotation axis.

The first image scan may be displayed with an overlay indicating the ugly duckling metric. The first image scan may also be displayed with an overlay indicating the ranked areas of interest.

The ugly duckling metric may be calculated using a portion of the first image scan. The ugly duckling metric may be displayed as a saliency map. The ugly duckling metric may be displayed as a body heatmap. The ranked areas of interest may be displayed as a montage.

In accordance with another aspect of the present invention there is provided a method of detecting skin conditions. In accordance with the method, a first image scan is captured, areas of interest of the first image scan are identified using a trained custom image segmentation model, wherein the trained custom image segmentation model is trained using a first plurality of image scans, each of the areas of interest are classified to correspond to skin conditions, wherein the skin conditions include at least one of eczema, hives, contact dermatitis, autoimmune skin conditions, bacterial infections, viral infections, fungal infections, burns, scars, grafts, childhood skin conditions, skin cancers, and psychosomatic skin disorders, each of the areas of interest are routed to at least one trained specialist model, the trained specialist model corresponding to one or more of the skin conditions, wherein the at least one trained specialist model is a deep neural network trained using a second plurality of image scans, the areas of interest are classified using the at least one trained specialist model, a deep feature layer of the at least one trained specialist model is extracted for each of the areas of interest, a skin condition metric is calculated for each of the areas of interest using the deep feature layer, the skin condition metric corresponding to a standardized scoring system for evaluating at least one of the skin conditions, the skin condition metric associated with each of the areas of interest is displayed in accordance with the first image scan, and ranked areas of interest identified in the first image scan are displayed, ranked in accordance with the classifications.

Each of the first image scan and the first and second pluralities of image scans may include a 360-degree view of at least a portion of a body part.

In accordance with another aspect of the present invention there is provided a skin condition detection system that includes an imaging device configured to capture a first image scan of a first user, a plurality of datasets that include a plurality of image scans of multiple users, a database configured to store the first image scan and the plurality of image scans, a trained image segmentation model, wherein the trained image segmentation model is trained using the plurality of image scans stored on the database, and is configured to identify areas of interest corresponding to classifications of skin conditions and to output segmented image data for each of the areas of interest, at least one trained specialist model configured to receive the segmented image data and to classify the segmented image data for each of the areas of interest for at least one of the skin conditions, and a display configured to display a skin condition metric based on a standardized scoring system for evaluating at least one of the skin conditions for each of the areas of interest, and ranked areas of interest identified in the first image scan ranked in accordance with the classified segmented image data.

The first image scan may include a 360-degree view of at least a portion of a body part. The plurality of image scans of multiple users may also include at least one 360-degree view of at least a portion of a body part. The plurality of image scans of multiple users may include images of at least one skin condition.

The at least one trained specialist model may be a deep convolutional neural network. The skin condition metric may be calculated using a deep feature layer of the deep convolutional neural network in accordance with the standardized scoring system. The at least one trained specialist model may correspond to one or more of the skin conditions.

The skin conditions may include at least one of eczema, hives, contact dermatitis, autoimmune skin conditions, bacterial infections, viral infections, fungal infections, burns, scars, grafts, childhood skin conditions, skin cancers, and psychosomatic skin disorders.

### BRIEF DESCRIPTION OF THE DRAWINGS

FIG. 1 is a schematic diagram of an imaging and diagnostic system in accordance with a preferred embodiment of the present invention;

FIG. 2 is a perspective view of a rotatable imaging device in accordance with another preferred embodiment of the present invention;

FIG. 3 is a functional block diagram of a body part detection system and method in accordance with a preferred embodiment of the present invention;

FIG. 4 is a functional block diagram of a scar, mark, and tattoo detection system and method in accordance with a preferred embodiment of the present invention;

FIG. 5 is a functional block diagram of a skin abnormality detection system and method in accordance with a preferred embodiment of the present invention;

FIG. 6 is a functional block diagram of a skin abnormality detection system and method in accordance with a preferred embodiment of the present invention;

FIG. 7 is a functional block diagram of a skin abnormality detection system and method in accordance with a preferred embodiment of the present invention;

FIG. 8 is a flowchart illustrating a body change detection method in accordance with a preferred embodiment of the present invention;

FIG. 9 is a flowchart illustrating a skin abnormality detection method in accordance with a preferred embodiment of the present invention; and

FIG. 10 is a flowchart illustrating a skin condition detection method in accordance with a preferred embodiment of the present invention.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

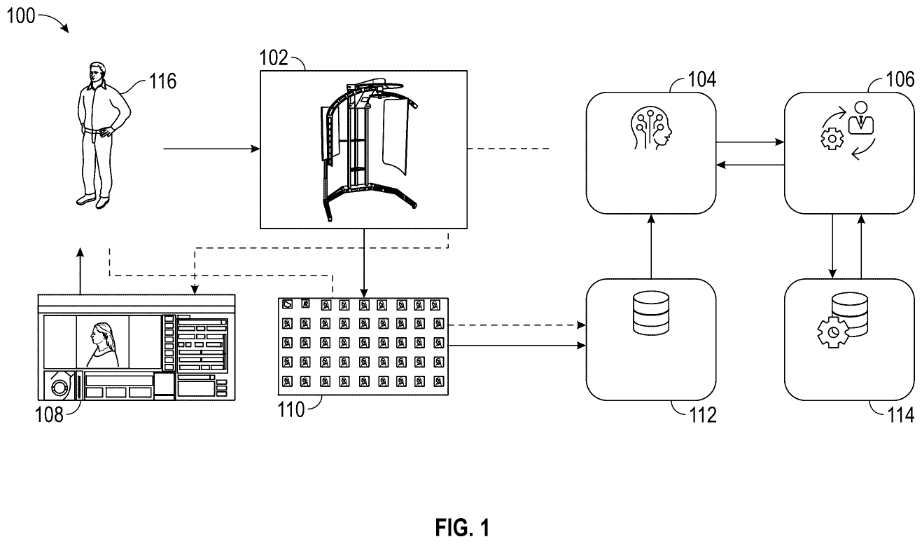

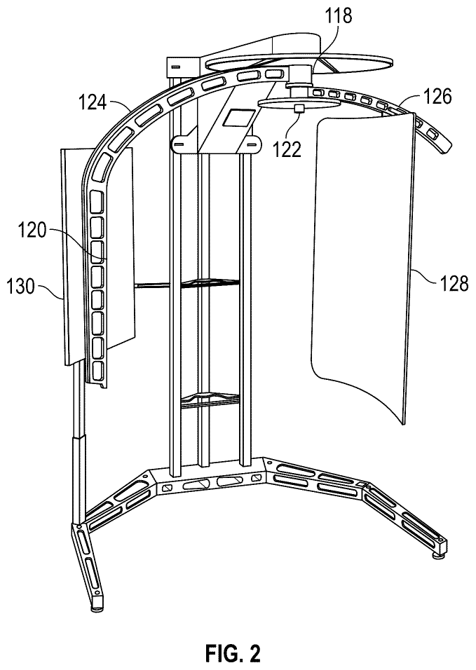

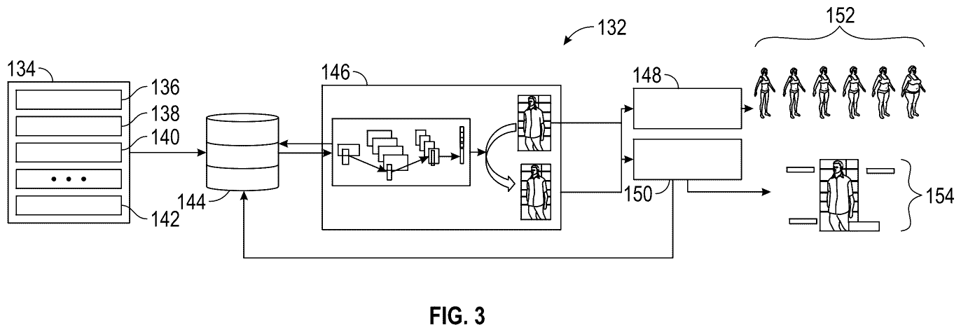

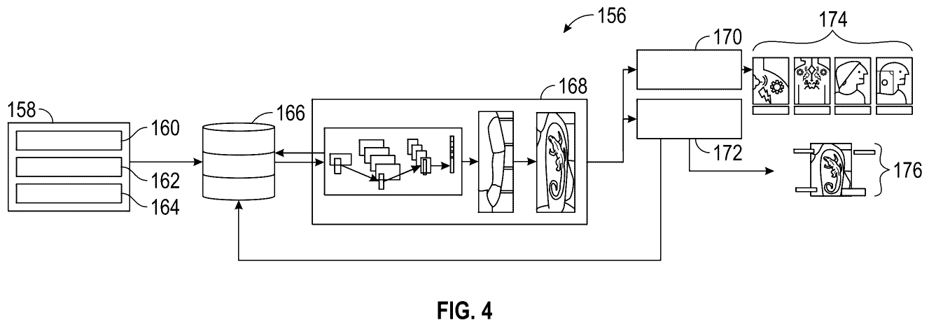

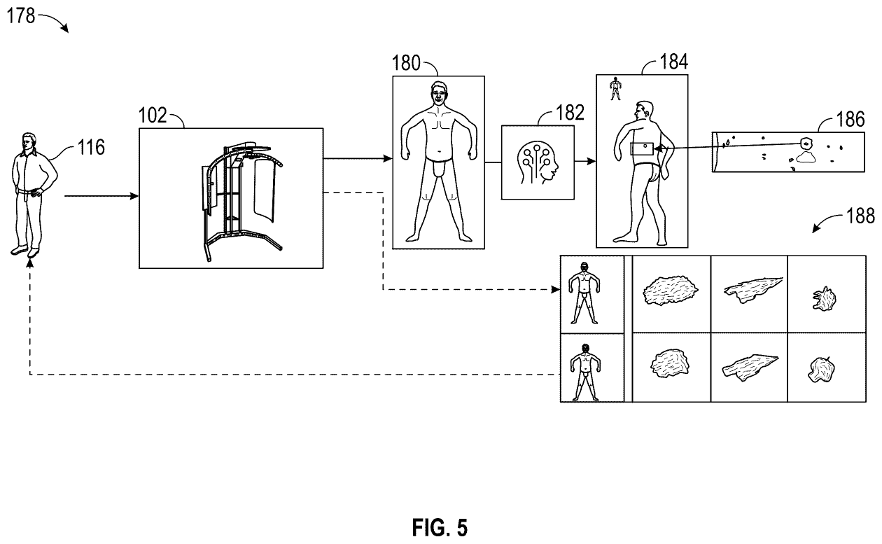

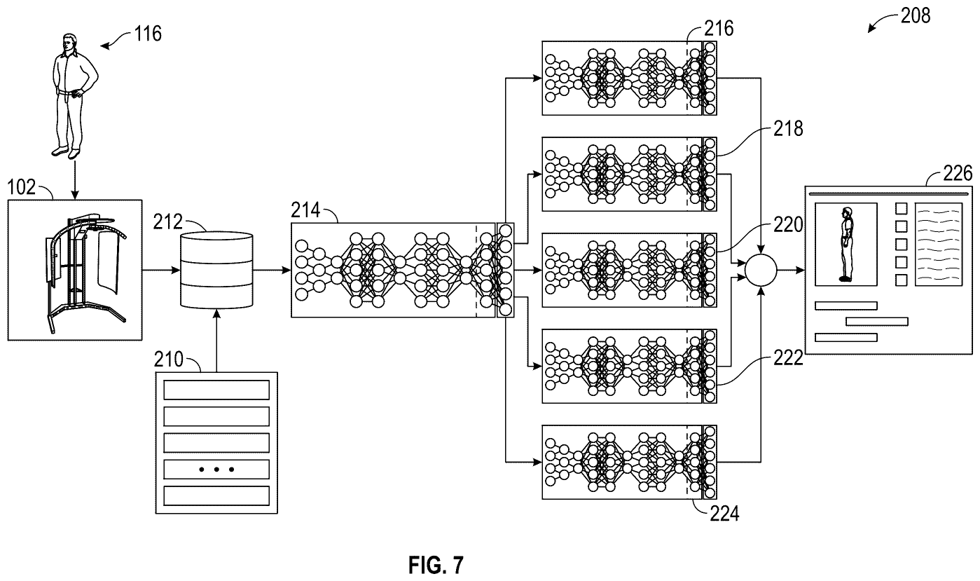

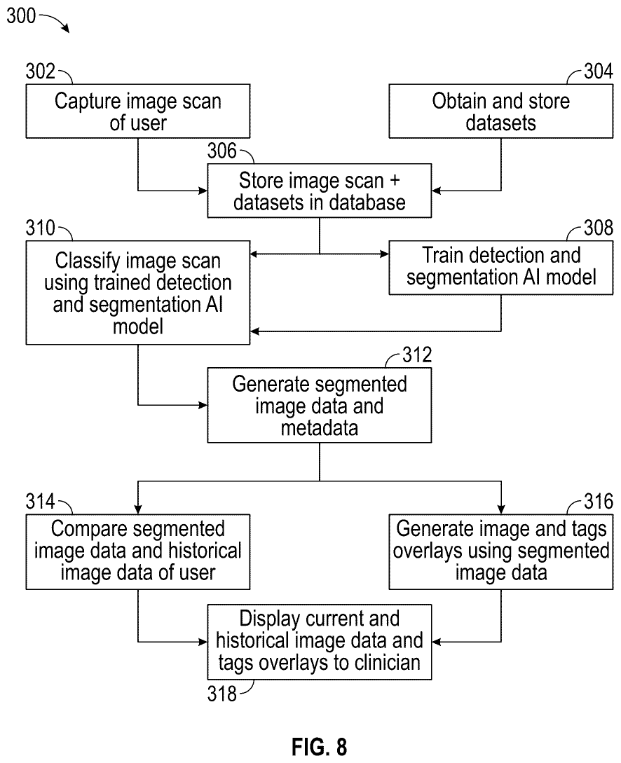

Referring now to the drawings, which are for purposes of illustrating the present invention and not for purposes of limiting the same, the drawings show devices and components (and related methods) therein in accordance with preferred embodiments of a body imaging and detection system and method. FIGS. 1-2 generally relate to an imaging and diagnostic system. FIGS. 3 and 7 generally relate to a body image detection system and method. FIGS. 4 and 8 generally relate to a scar, mark, tattoo, and lesion detection system and method. FIGS. 5 and 9-10 generally relate to a skin abnormality or skin condition detection system and method.

Referring now to FIG. 1, FIG. 1 is a schematic diagram of an imaging and diagnostic system in accordance with a preferred embodiment of the present invention. The system 100 may include an imaging device 102, an AI training and tuning module 104, a management module 106, a user interface 108, a raw data output 110, a data generation module 112, and a database server 114. Also referenced is a user 116 of the system 100.

The imaging device 102 may be a 360-degree imaging device. In a preferred embodiment, the imaging device 102 is an oVio360® imaging device as described in U.S. Pat. No. 10,171,734, the entirety of which is incorporated by reference in its entirety. The imaging device may have a first arm that rotates around a user capturing images and a second arm that is simultaneously positioned 180-degrees away from the first arm to block light from behind the user.

The imaging device 102 may include one or more cameras, one or more scanners, and/or employ three-dimensional (3D) imaging techniques. The imaging device 102 may capture a 360-degree view image (e.g., 360-degree image scan). The imaging device 102 may capture a plurality of images at various angles around a subject to generate the 360-degree view image. For example, the imaging device 102 captures 360 images at each of the degrees in a circumference around the subject. The images may be displayed on the display of the system 100 via the user interface 108. The captured images may be 3D. Each 3D image may be separated into two-dimensional (2D) images captured from various angles around a user that were used in generating the 3D image. The system is usable, for example, for helping diagnose cancer or other issues related to moles or other skin blemishes, lesions or the like, for before and after imaging in plastic surgery, to identify or quantify body change characteristics or for identification. A person of ordinary skill in the art would recognize that the aforementioned examples are non-limiting and that the present invention includes other examples or applications.

The imaging device 102 may generally include a memory such as a random-access memory (RAM), a disk, a flash memory, an optical disk drive, a hybrid memory, or any other storage medium that can store data. The memory may store program code that is executable by a processor of the imaging device 102. The memory may store data in an encrypted or any other suitable secure form, for example, on a database. In some examples, the memory may be a memory of a remote server.

The imaging device 102 also may generally include a processor configured to execute machine-readable instructions. In some examples, there may be a plurality of processors. In some examples, the processor may be a microprocessor or a microcontroller. The processor may be electronically coupled to one or more other components of the system 100, wirelessly or wired. The processor may be integrated with the imaging device 102 or another component of the system 100. In some examples, the processor may be a processor of a remote server in electronic communication with the system 100.

The system 100 may also include a display such as a liquid crystal display (LCD), a light-emitting diode (LED) display, an organic light emitting diode (OLED) display, a plasma display, a cathode-ray tube (CRT) display, a digital light processing display (DLPT), a microdisplay, a projection display, or any other display appreciated by one of ordinary skill in the art. The display may display user interfaces, text, images, and/or the like. For example, the display is integrated with the user interface 108.

The system 100 may also include an input device such as buttons and/or a touchscreen, and hence may be integrated with the display. In other examples, the input device may include knobs, dials, keys, pads, a mouse, a microphone, a camera, and/or the like. The input device may be used to provide user instructions to the processor and navigate a user interface displayed by the display.

The system 100 may store body data in the database server 114. The body data may include one or more images associated with one or more body parts and/or one or more portions of body parts. The system 100 may compare the captured image to the images included in the body data to identify body parts and/or portions of body parts. A display may display the captured image and overlay each image or portion of an image indicating an identified body part and/or portion of body part.

The system 100 may further store skin change data in the database server 114. The skin change data may include images of changes on skin. Skin changes may include moles, freckles, tan lines, wrinkles, cuts, bruises, lesions, scars, marks, tattoos, bumps, lumps, and/or the like. The system 100 may compare the captured image to the images of the skin change data to identify a change on the subject's skin. For example, the system 100 may identify that the user has a tattoo. Additionally, the system 100 may determine one or more physical features of the skin change. A physical feature may include a location, position, size, shape, etc. of the skin change. For example, the system 100 may identify that the tattoo has a lizard shape and is located on the subject's left shoulder. The processor may identify the subject based on the skin change, or make an identification of the subject based on the location, position, size, shape, etc. of a known historical skin change.

In another embodiment, the system 100 may capture an image of a user, a patient, a subject and/or the like. The image may be a full body picture that includes an entirety of the subject's body or self. In some examples, the image may only include a portion of the body, a body part, or a portion of a body part. Once the image is captured, the image may be saved on the database server 114. The database server 114 may store user image history. The user image history may include a previously captured image or images of the user, subject, or the like. The database server 114 may store a user image history for multiple users. The system 100 may compare the images in the database to the most recently captured image to determine an identity of the subject. Once the identity of the subject is determined, the system 100 may limit further analysis to the user image history for the identified subject. The system 100 may analyze the previously captured images and the metadata and make a comparison between the previously captured images and the most recently captured image. Based on the comparison, the processor may determine a change in the body, the body portion, the body part, or the portion of the body part. For example, the system 100 may determine that the user has gained weight, has a new hairstyle, and has undergone nose surgery (i.e., rhinoplasty).

The AI training and tuning module 104 is preferably implemented in executable software stored on the system 100. The module 104 preferably performs the training and tuning (e.g., fine-tuning) of the various AI models disclosed herein. The module 104 preferably receives training data from the data generation module 112. A person of ordinary skill in the art will appreciate that, as described herein, there are a variety of manners and methods of training and tuning a model using images and is non-limiting. The management module 106 is preferably implemented in executable software stored on the system 100 and is configured to allow an AI specialist to train and tune AI models and store and retrieve data from the database server 114. The management module 106 may also be configured to execute trained AI models for use with the system 100 in accordance with the systems and methods disclosed herein. The user interface 108 is preferably coupled to the data generation module 112 and the imaging device 102 and is configured to conduct image scans of a subject. The user 116 is preferably the operator of the system 100 and uses the user interface 108 to interact with the system. The user 116 may also in certain embodiments interact with the management module 106. The user interface 108 is also configured to generate the raw data output 110 that is output from the imaging device 102.

The data generation module 112 is preferably implemented in executable software stored on the system 100. The data generation module 112 may be configured to perform functions to pre-process images and related metadata for use by the AI training and tooling module 104. For example, images may need to be reduced in size, cropped, rotated, or otherwise manipulated prior to being used in the training and/or tuning process. Other functions of the data generation module 112 are within the scope of the present invention and generally relate to the nature and type of training, tuning, or model desired to be generated.

The database server 114 is preferably a relational database (as disclosed herein) and is configured to store, organize, and receive images and relational metadata. Other database architectures are within the scope of the present invention.

Referring now to FIG. 2, FIG. 2 is a perspective view of a rotatable imaging device in accordance with another preferred embodiment of the present invention. The rotatable imaging device 102 preferably includes a rotating unit 118, an imaging camera 120, an alignment camera 122, a first arm 124, a second arm 126, a backdrop 128, and an alignment monitor 130.

The rotating unit 118 includes the first arm 124 configured to couple with the imaging camera 120 (e.g., the imaging camera 120 is mounted on the first arm 124) and the second arm 126 configured to couple with the backdrop 128 (e.g., the backdrop 128 is mounted on the second arm 126). Preferably, the imaging camera 120 is configured to capture an image scan, as described herein. The image scan may be of a subject's whole body or of a portion thereof. The backdrop 128 preferably provides consistent lighting and position for the image scan to be captured. The first arm 124 and the second arm 126 together form a horizontally oriented boom that rotates about a rotation axis with the imaging camera 120 on one side (e.g., on the first arm 124 side) and the backdrop/screen 128 on the other side (e.g., on the second arm 124 side).

The rotating unit 118 is preferably rotatable between a home position and a finish position about a rotation axis such that the imaging camera 120 captures an image scan.

The alignment camera 122 is preferably positioned above a subject and preferably is co-axial with a pivot axis (e.g., rotation axis) of the imaging camera 120 and the backdrop 128. The alignment monitor 130 is used in conjunction with the alignment camera 122 to aid in the alignment and centering of the subject so that consistent image scans may be captured. For example, the alignment monitor 130 includes a graphical depiction of the subject's body (e.g., head, shoulders, or the like) and an alignment image from the alignment camera 122 so that the subject's body is positioned and centered about the pivot axis, which facilitates consistent image scans across multiple scans of the subject, and other subjects, that are utilized in the method and system disclosed herein.

In a preferred embodiment, the components for rotating the rotating unit 118 (e.g., a motor control unit, computer components, motor, cooling fan, etc.) are housed within, connected to or shrouded by structural elements of the imaging device 102, as appreciated by one of ordinary skill in the art.

Referring now to FIG. 3, FIG. 3 is a functional block diagram of a body part detection system and method in accordance with a preferred embodiment of the present invention. In a preferred embodiment the system 132 includes datasets 134, a database 144, a detection and segmentation AI model 146, a body part analysis engine 148, and a body part tagging engine 150. Preferably, the system 100 is utilized to perform the body part detection method associated with the body part detection system 132. Body part detection may be executed by a trained AI. The system 132 may be provided datasets of various known body parts or images of identified body parts in full body images. As such, the system 132 may learn to associate a certain body part image with that body part. The system 132 may continue to learn and increase accuracy as new data is obtained through newly captured images added to the database 144.

The datasets 134 are preferably stored to be retrieved or accessed in connection with AI model training. For example, the datasets include an arm dataset 134, a head dataset 136, a foot dataset 138, and an “N” body part dataset 140. One skilled in the art would appreciate that the “N” body part dataset 140 is a placeholder for various other types of body parts and is non-limiting. The datasets 134 include images of body parts, preferably captured using the imaging device 102. In another embodiment, the datasets 134 include images from other public or non-public datasets.

The datasets 134 are preferably pre-processed by manually tagging and segmenting the images of body parts before being stored in the database 144. For example, the database 144 is a relational database such as MySQL, PostgreSQL, Microsoft SQL Server, and Oracle Database, or the like. Thus, images, metadata, and training/testing data may be stored on the database 144. The images preferably are relationally associated with metadata regarding the image. For example, the manual tagging process may associate an image with a specific body part; i.e., an arm, a head, a hand, etc., and store structured metadata associated with that tag in the database 144 related to that image. In another embodiment, because the datasets 134 are segmented by body part, it may be suitable to automatically or manually tag each of the images in the database 144 with metadata specifying the body part. In yet another embodiment, images may already be associated with metadata and stored in the database 144. For example, the tagging engine 150 may provide tagged images to the database 144 for storage. It will be appreciated to a skilled person that the dataset 134 may consist of image scans of various body parts, each image scan comprising 360 (or greater or fewer) images. In a preferred embodiment, the database 144 includes images relationally associated with tags/metadata to both train the detection and segmentation AI model 146 as well as to utilize the model 146 to detect and segment input images for later use by the body part analysis engine 148.

The detection and segmentation AI model 146 is preferably a deep neural network configured to receive images from the database 144 and segment portions of the images detected as being body parts. For example, the model 146 is an image segmentation model configured to classify each pixel in an image. The images depicted in the model 146 of FIG. 3 illustrate taking a raw image and mapping each pixel to a class representing a body part. In an embodiment, the model 146 is a pre-trained Deep Convolutional Neural Network (DCNN) that is fine-tuned with data from tens of thousands of images captured from the imaging device 102. In other embodiments, the model 146 is a completely custom image segmentation model, or trained with greater or fewer images (e.g., 5,000, 100,000, etc.). A person of ordinary skill in the art would understand that there are a variety of ways to train an image segmentation model without departing from the scope of the present invention. In yet other embodiments, the model 146 is an object detection model or other model configured to identify, detect, and/or segment body parts in an image.

In a preferred embodiment, the detection and segmentation AI model 146 outputs segmented image data (e.g., body part data) to the body part analysis engine 148. The segmented image data may include position, size, shape, physical feature, or other information or metadata about a body part. The output from the model 146 may include the metadata output from the model 146, image-based feature construction of features of a body part, or the entire body of a subject, and an integration and conversion of feature data. The analysis engine 148 preferably compares the segmented image data with historical images and/or image data of the same subject. The comparison may also compare the segmented image data to images and/or image data of other subjects. The comparison may identify changes in body parts of the subject, such as a weight loss or gain, a new hairstyle, and/or rhinoplasty of the nose. For example, as shown in the body change graphic 152, a subject's overall body shape may be graphically displayed. In other embodiments, the graphic 152 is a body part or feature of the subject. The graphic 152 may be displayed as one or more images of the user image history along with the most recently captured image. For example, as shown in FIG. 3, the images comprising the graphic 152 may be displayed chronologically to demonstrate a weight gain of the subject over a time period. The output of the analysis engine 148 is preferably used by the subject or a clinician in identifying changes to the body parts or features of a subject and drawing clinical conclusions therefrom.

In a preferred embodiment, the detection and segmentation AI model 146 also outputs segmented image data (e.g., body part data) to the body part tagging engine 150. In an embodiment, a portion of the segmented image data is output to the tagging engine 150. The tagging engine 150 preferably uses the segmented image data or portion thereof to create tags for each of the detected and segmented body parts and outputs display data to be graphically displayed as a labeled and tagged image 154. The labeled and tagged image 154 depicted in FIG. 3 includes text labels that annotate the image of the subject and is preferably graphically displayed next to the body change graphic 152. For example, the text labels of the labeled and tagged image 154 specifies the body change or body feature. As with the analysis engine 148, the output of the tagging engine 150 is preferably used by the subject or a clinician in identifying changes to the body parts or features of a subject and drawing clinical conclusions therefrom.

Referring now to FIG. 4, FIG. 4 is a functional block diagram of a scar, mark, and tattoo (SMT) detection system and method in accordance with a preferred embodiment of the present invention. In a preferred embodiment the system 156 includes datasets 158, a database 166, a detection and segmentation AI model 168, an SMT analysis engine 170, and an SMT tagging engine 172. Preferably, the system 100 is utilized to perform the SMT detection method associated with the SMT detection system 156. SMT detection may be executed by a trained AI. The system 156 may be provided datasets of various scars, marks, or tattoos, or images of scars, marks, or tattoos in full body images. As such, the system 156 may learn to associate a certain body part image with that body part. The system 156 may continue to learn and increase accuracy as new data is obtained through newly captured images added to the database 166.

The datasets 158 are preferably stored to be retrieved or accessed in connection with AI model training. For example, the datasets include a scar dataset 160, a mark dataset 162, and tattoo dataset 164. The datasets 158 include images of scars, marks, and tattoos, preferably captured using the imaging device 102. In another embodiment, the datasets 158 include images from other public or non-public datasets.

The datasets 158 are preferably pre-processed by manually tagging and segmenting the images of scars, marks, and tattoos before being stored in the database 166. For example, the database 166 is a relational database such as MySQL, PostgreSQL, Microsoft SQL Server, and Oracle Database, or the like. Thus, images, metadata, and training/testing data may be stored on the database 166. The images preferably are relationally associated with metadata regarding the image. For example, the manual tagging process may associate an image with a specific scar, mark, or tattoo associated with a portion of the subject's body and store structured metadata associated with that tag in the database 166 related to that image. In another embodiment, because the datasets 158 are segmented by scars, marks, and tattoos, it may be suitable to automatically or manually tag each of the images in the database 166 with metadata specifying the body part. In yet another embodiment, images may already be associated with metadata and stored in the database 166. For example, the tagging engine 172 may provide tagged images to the database 166 for storage. It will be appreciated to a skilled person that the dataset 158 may consist of image scans of various body parts, each image scan comprising 360 (or greater or fewer) images. In a preferred embodiment, the database 166 includes images relationally associated with tags/metadata to both train the detection and segmentation AI model 168 as well as to utilize the model 168 to detect and segment input images for later use by the SMT analysis engine 170.

The detection and segmentation AI model 168 is preferably a deep neural network configured to receive images from the database 166 and segment portions of the images detected as being scars, marks, or tattoos. For example, the model 168 is an image segmentation model configured to classify each pixel in an image. The images depicted in the model 168 of FIG. 4 illustrate taking a raw image and mapping each pixel to a class representing a scar, mark, or tattoo on a body part. In an embodiment, the model 168 is a pre-trained Deep Convolutional Neural Network (DCNN) that is fine-tuned with data from tens of thousands of images captured from the imaging device 102. In other embodiments, the model 168 is a completely custom image segmentation model, or trained with greater or fewer images (e.g., 5,000, 100,000, etc.). A person of ordinary skill in the art would understand that there are a variety of ways to train an image segmentation model without departing from the scope of the present invention. In yet other embodiments, the model 168 is an object detection model or other model configured to identify, detect, and/or segment body parts in an image.

In a preferred embodiment, the detection and segmentation AI model 168 outputs segmented image data (e.g., SMT data) to the SMT analysis engine 170. The segmented image data may include position, size, shape, physical feature, or other information or metadata about a scar, mark, or tattoo on a body part. The output from the model 168 may include the metadata output from the model 168, image-based feature construction of features of a body part including a scar, mark, or tattoo, or the entire body of a subject depicting all scars, marks, and tattoos, and an integration and conversion of feature data. The analysis engine 170 preferably compares the segmented image data with historical images and/or image data of the same subject. The comparison may also compare the segmented image data to images and/or image data of other subjects. The comparison may identify new scars, marks, or tattoos, and/or identify the subject base on the SMT data. For example, as shown in the SMT graphic 174, a subject's image and a “match” image are graphically displayed to illustrate that the scar, mark, or tattoo detected matches an image from the database 166, suggesting that the subject whose image(s) were obtained is an individual that matches the historical images of the database 166. In other embodiments, the graphic 174 is a body part or feature of the subject including the scar, mark, or tattoo. The output of the analysis engine 170 is preferably used by the subject, a clinician, or other user in identifying the subject and drawing conclusions therefrom.

In a preferred embodiment, the detection and segmentation AI model 168 also outputs segmented image data (e.g., SMT data) to the SMT tagging engine 172. In an embodiment, a portion of the segmented image data is output to the tagging engine 172. The tagging engine 172 preferably uses the segmented image data or portion thereof to create tags for each of the detected and segmented scars, marks, and tattoos and outputs display data to be graphically displayed as a labeled and tagged image 176. The labeled and tagged image 176 depicted in FIG. 3 includes text labels that annotate the image of the subject and is preferably graphically displayed next to the SMT graphic 174. For example, the text labels of the labeled and tagged image 176 specifies the scar, mark, or tattoo associated with the subject's body part(s). As with the analysis engine 170, the output of the tagging engine 172 is preferably used by the subject, a clinician, or other user in identifying the subject and drawing conclusions therefrom.

Referring now to FIG. 5, FIG. 5 is a functional block diagram of a skin abnormality detection system and method in accordance with a preferred embodiment of the present invention. In a preferred embodiment, the system 178 includes an imaging device 102, a 3D body image scan 180, a trained AI model 182, a body heatmap 184, a scar, mark, tattoo, and lesion (SMTL) graphic 186, and a lesion graphic 188. Preferably, the system 100 is utilized to perform the skin abnormality detection method associated with the skin abnormality detection system 178. Skin abnormality as disclosed herein includes SMT/SMTL detection and skin lesion detection, but a skilled artisan will appreciate that the system and method disclosed herein is not limited to detecting SMT/SMTL and skin lesion detection. Skin abnormality detection may be executed by a trained AI. The system 178 may be provided datasets of various scars, marks, tattoos, skin lesions, or images of scars, marks, tattoos, or skin lesions in full body images. As such, the system 178 may learn to associate a certain body part image with that body part. The system 178 may continue to learn and increase accuracy as new data is obtained through newly captured images added to a database.

Similarly to the SMT detection system 156, the skin abnormality detection system 178 in a preferred embodiment incorporates the system elements of the SMT detection system 156 such as the datasets 158, the database 166, the detection and segmentation AI model 168, the SMT analysis engine 170, and the SMT tagging engine 172. Description of those system components are described in detail herein and are not repeated for purposes of brevity.

The system 178 also preferably utilizes an imaging device 102, as described in detail herein. The 3D body image scan 180 is a preferred output of the imaging device 102, as described herein as the output of the system 100. Other imaging devices 102 may be utilized to output the 3D body image scan 180 without departing from the scope of the present invention.

The trained AI model 182 in a preferred embodiment in a preferred embodiment also includes a deep neural network configured to receive the 3D body image scan 180 (and other images) and segment portions of the images detected as being scars, marks, tattoos, or skin lesions. Thus, the designation SMT may include skin lesions, as described herein. For example, the model 182 is an image segmentation model configured to classify each pixel in an image. The model 182 may take a raw image and map each pixel to a class representing a scar, mark, tattoo, or skin lesion on a body part. In an embodiment, the model 182 is a pre-trained Deep Convolutional Neural Network (DCNN) that is fine-tuned with data from tens of thousands of images captured from the imaging device 102 (e.g., images such as the 3D body image scan 180). In other embodiments, the model 182 is a completely custom image segmentation model, or trained with greater or fewer images (e.g., 5,000, 100,000, etc.). A person of ordinary skill in the art would understand that there are a variety of ways to train an image segmentation model without departing from the scope of the present invention. In yet other embodiments, the model 182 is an object detection model or other model configured to identify, detect, and/or segment body parts in an image.

The body heatmap 184 is a preferred output of the model 182 and/or a SMTL analysis engine, which is similar to the function of the SMT analysis engine 170. For example, the body heatmap 184 displays scar, mark, tattoo, and skin lesion data overlayed on an image of a subject or the subject's body part(s). The SMTL graphic 186 depicted as a callout to the body heatmap 184 and preferably includes a blown-up portion of the image and view of a detected scar, mark, tattoo, or skin lesion.

The lesion graphic 188 may display one or more images of the user image history along with the most recently captured image. For example, as shown in FIG. 5, the previously captured images where moles or other skin lesions were detected may be shown simultaneously with the recently captured images where lesions are currently being detected. The differences in the physical features of the lesions, such as size, shape, and color, may be observed by a qualified medical professional to diagnose and treat the user. In some examples, the system 178 may compare a captured image (e.g., the lesion graphic 188) with a database of images depicting lesions that were assigned an ulcerative dermatitis score and/or a nonsuspicious pigmented lesion (NSPL) or a suspicious pigmented lesion (SPL) classification. The system 178 may assign lesions depicted in the legion graphic 188 an ulcerative dermatitis score and/or a NSPL or a SPL classification by comparing the captured image to those of the database (e.g., the database server 114, the database 166, or other suitable database or remote server). The ulcerative dermatitis score may be based on a variety of factors that may include the number of lesions, the number of scratches over or around the lesions, character of the lesions (e.g., excoriations, small punctuate crust, coalescing crust, erosion, ulceration), and/or the length of the lesions. The NSPL classification may include a type A classification of low priority and a type B classification of medium priority. The SPL, NSPL, or other classifications may be implemented in a DCNN, or the equivalent, to accurately and efficiently classify skin lesions. The identification of areas of interest on a body part of a subject may be determined using ugly duckling (UD) criteria to identify potentially cancerous skin lesions, including melanoma. A person of ordinary skill in the art would be familiar with UD criteria and how it assists a clinician in identifying a mole or skin lesion that looks different from the other moles or skin lesions on a subject's body or body part(s).

The skin abnormality detection system 178 may be used in practice to diagnose or help diagnose skin cancer. In use, one or more scans or 360-degree image scans may be taken of the subject or portions of the subject's body. The system may identify moles or other lesions and classify them based on the characteristics discussed above (e.g., based on a database of moles or lesions, or classification system) to help determine if any of the moles or lesions may be cancerous, suspect, or suitable for biopsy. This type of scan can be done quickly and may be reduce the amount of time a dermatologist needs to examine individual moles or lesions of a patient. The scan may also assist in eliminating false positives, providing the dermatologist additional information from which to order further testing, biopsies, or the like. The images may then be stored so that the skin lesions can be compared at future visits. The body heatmap 184 and/or the lesion graphic 188 may be used by a subject, clinician, or other user to provide feedback and derive conclusions therefrom.

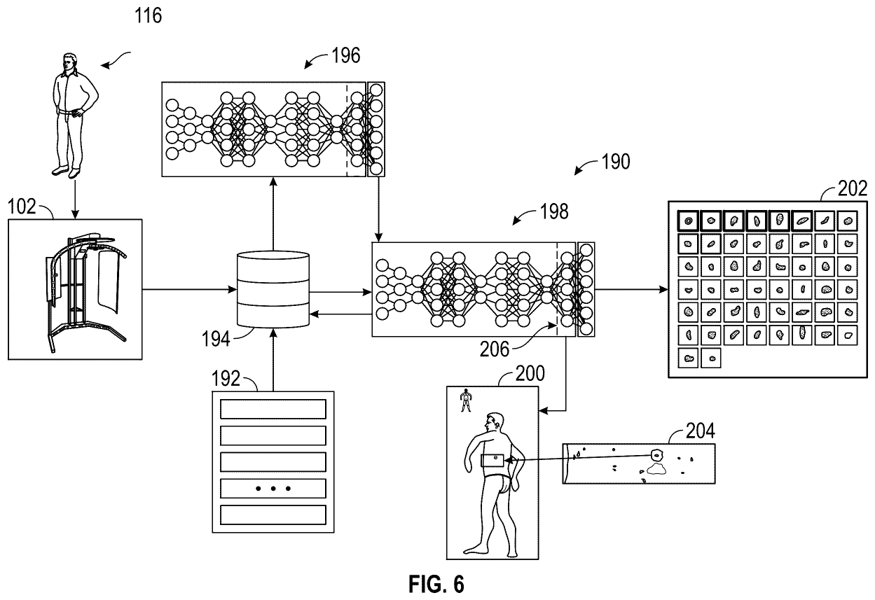

Referring now to FIG. 6, FIG. 6 is a functional block diagram of a skin abnormality detection system and method in accordance with a preferred embodiment of the present invention. In a preferred embodiment, the system 190 includes an imaging device 102, datasets 192, a database 194, a trained image segmentation model 196, a DCNN 198, a body heatmap 200, a lesion montage 202, a labeled lesion graphic 204, and a deep feature layer 206.

The imaging device 102 has been described extensively herein. In an embodiment where the imaging device 102 is a 360-degree imaging device, the system 190 is capable of leveraging the unique photography capabilities of an oVio360 capture device. For example, each image scan contains 360 individual images with highly consistent lighting, position, and backdrop. Each single 360-degree imaging device is preferably capable of capturing and uploading tens of thousands of 2048×2048 pixel images to the datasets 192 or the database 194, either on an on-premises or cloud-based storage. A large, real-world dataset is therefore achievable that standard image datasets cannot replicate. The system 190 preferably uses the images of the image scans to fine-tune a pre-trained DCNN and eliminate or reduce noisy, inconsistent backgrounds often found in standard images taken in a primary care practice (PCP) setting, in telemedicine settings, or a subject's images taken with non-standard photography equipment.

In addition, a 360-degree image device is capable of rapidly transferring data at reduced cost to efficiently create unique, novel datasets for fine-tuned CNNs based on dermatology screenings without relying on synthetic data or data augmentation. The use of real world, consistent data provided by the 360-degree image device (or devices) can therefore outperform models built on public image repositories, while offering lower cloud computing costs through reduction in neural network complexity. The datasets used to fine-tune CNNs may also be further enhanced by incorporating patient data (e.g., Electronic Medical Records (EMR)) to accommodate expanded risk assessment using key demographic data such as age, ethnicity, family history, and genetic screening information. For example, EMR data can be used to train new deep learning models to determine patient risk level, adding an extra layer of confidence to the initial triage. A CNN fine-tuned with EMR data may include genetic markers that provide further screening capabilities for the system 190 by analyzing skin abnormalities of such subjects and comparing them to similarly situated subjects with those same genetic markers.

For example, the EMR data may be mined or analyzed for demographic information and stored in one or more datasets. Thus, the datasets 192 preferably include 360 images of 360-degree image scans, mined or analyzed EMR data, and related metadata. A person of ordinary skill in the art would recognize that the datasets 192 may include other data, including standard datasets or public image repositories. As described extensively herein, the datasets 192 may themselves be stored on a database, stored on the database 194, or a combination thereof. In addition, the data of the datasets 192 may be further analyzed, mined, or processed before storing images and related metadata or other relational data on the datasets 192 and/or the database 194.

The trained image segmentation model 196 is preferably an image segmentation model trained using images or image scans with related metadata from the 360-degree image device (or devices). A person of ordinary skill in the art would recognize that an image segmentation model may be implemented in a CNN. For example, the model 196 is an image segmentation model configured to classify each pixel in an image. The model 196 may include taking a raw image and mapping each pixel to a class representing a particular class of skin lesions or to skin abnormalities generally. In an embodiment, the model 196 is a pre-trained DCNN that is fine-tuned with data from tens of thousands of images captured from the imaging device 102. In other embodiments, the model 196 is a completely custom image segmentation model, or trained with greater or fewer images (e.g., 5,000, 100,000, etc.). The trained image segmentation model may be trained using multiple image scans each comprising a 360-degree view of at least a portion of a body part. A person of ordinary skill in the art would understand that there are a variety of ways to train an image segmentation model without departing from the scope of the present invention. In yet other embodiments, the model 168 is an object detection model or other model configured to identify, detect, and/or segment body parts in an image.

Once the model 196 classifies a particular image as containing a skin abnormality or a particular skin lesion, the system 190 identifies areas of interest of the image scan. The system 190 preferably crops and stores each area of interest for further processing and analysis. As shown in FIG. 6, the classification results, as well as the cropped areas of interest, may be stored in the database 194 for later retrieval by the DCNN 198 or may be routed directly to the DCNN 198 for further processing and analysis.

The model 196 provides improvements to standard techniques of detecting blobs, such as using Scale-Invariant Feature Transform (SIFT) and/or Laplacian of Gaussian (LoG) algorithms to detect blobs or abnormalities. The improvement in detection may assist in eliminating or reducing false positives and provide practical results for the clinician in a PCP environment.

The model 196, as described herein, preferably includes 360 images from a 360-degree image scan. This unique full-body dataset allows for extended comparisons for intra-patient lesion saliency. For example, the detection of individual skin abnormalities may be enhanced by eliminating or reducing false positives that inconsistently appear when analyzed from several angles. The 360 images preferably are taken from each of the 360 degrees about the subject and present multiple non-overlapping angles of the same skin abnormality. This allows for deep feature comparison across the subject's entire body or portions thereof.

In an embodiment, the model 196 is a shallow CNN trained exclusively on data from 360-degree image scans of the imaging device 102. Shallow CNNs may require less data to train and may present easier targets for leveraging explainable AI (xAI) techniques, which may allow humans to more confidently interpret AI outputs. As more fully described herein in connection with FIGS. 7-10, these improvements are suitable to rapidly develop similar models for other rapid detection problems, within dermatology and beyond.

The DCNN 198 is preferably a DCNN trained with image scans each including a 360-degree view of at least a portion of a body party. The DCNN 198 preferably classifies the areas of interest identified by the model 196 in accordance with classifications such as SPL, medium-priority NSPL Type B (NSLP-B), and low-priority NSPL Type A (NSLP-A) classifications. In addition, the classifications may include backgrounds, skin edges, and bare skin sections. For example, the output classifications of the DCNN 198 correspond with the five neural nodes depicted as being output from the DCNN 198 in the graphic of FIG. 6.

The SPL classification may be trained using a combination of public-access dermatology repositories, web scraping outputs, and identified clinical images. The SPL classification may also be trained exclusively or partially on images obtained from 360-degree image scans of the imaging device 102. The SPL dataset may consist of melanomas stages 0 to IV, squamous cell carcinomas, and basal cell carcinomas where biopsy or excision is indicated. The NSPL-A and NSPL-B datasets used for training the DCNN 198 may be aggregated from distinct pigmented lesion subtypes indicating that low- or medium-priority management is indicated. For example, the NSPL-A dataset may be aggregated from nine pigmented lesion subtypes (e.g., Junctional nevus, Combined nevus, Congenital nevus, Dermal nevus, Dermatofibroma, Seborrheic keratosis, Acrochordon, Cherry angioma, and Lentigo) where low-priority management is indicated, and the NSPL-B dataset may be aggregated from nine pigmented lesion subtypes (e.g., Melanocytic nevus, Drysplastic nevus, Blue nevus, Clark nevus, Melanosis, Recurrent nevus, Reed Spitz nevus, Congential nevus, and Other non-cancer), where dermatological referral or follow-up are indicated to better assess the subject's risk of skin cancer. The background images may span a variety of non-skin objects such as those commonly found in PCP or home care settings, and may be excluded as a classification from the DCNN 198 when using 360-degree image scans captured by the imaging device 102. The skin edge, bare skin, NSPL-A, NSPL-B, and SPL classes may encompass a range of Fitzpatrick skin tones, types I to VII. The non-lesion related classes of backgrounds, skin edge, and bare skin may be included in the training dataset to further train the DCNN 198 to be capable of discriminating pigmented lesions from other features commonly observed in images obtained in the dermatological setting. A person of ordinary skill in the art would understand that other classifications and datasets are within the scope of the present invention.

The lesion montage 202 is preferably a graphical display of lesions detected by the DCNN 198. The lesion montage 202 is preferably generated using output results of the DCNN 198 to identify SPLs and UD criteria analysis to identify potential SPLs that looks different from the other moles or skin lesions on a subject's body or body part(s). The UD criteria analysis may include a saliency map that compares the level of difference of skin abnormalities to the others identified in the same image or portion thereof. The saliency map may be a non-DCNN saliency map created through lesion segments collaged into an inconspicuous (e.g., non-salient) synthetic background created by averaging the original image. The non-DCNN saliency map may be sensitive to the presence of fabrics, backgrounds, and other features found in a PCP environment; the use of 360-degree image scans captured by the imaging device 102 may alleviate this sensitivity and present better results than when using inconsistent, PCP or home care images that more likely include these other features. The saliency map may also be a DCNN saliency map that utilizes a deep feature layer 206 of the DCNN 198 in conducting the UD criteria analysis and generate the saliency map. The deep feature layer 206 preferably is a last layer, or close to a last layer, of the output of the DCNN 198. The deep feature layer 206 preferably includes the deep features from the DCNN 198 of the classifications. A saliency score may be calculated to generate the saliency map. For example, the saliency score is a patient-dependent metric of pigmented lesion oddness calculated using the geometric distance of deep features from the DCNN from all skin abnormalities of a single subject or patient. The saliency map in a preferred embodiment is an image of a subject or portion thereof with a saliency overlay highlighting or color-coding skin abnormalities for use by clinicians when conducting referral decisions. In another embodiment, the saliency map includes a t-distributed stochastic neighbor embedding (t-SNE) graph showing the clustering of all skin abnormalities in the field of view (e.g., the image or portion thereof) of the subject.

A person of ordinary skill in the art would understand that the classifications of the DCNN 198 may, or may not, classify background or other non-lesion images and may focus solely on SPLs and NSPLs, as described herein.

In another embodiment, the lesion montage 202 is generated without the use of UD criteria analysis and identifies SPLs and NSPLs in the lesion montage based on the output classifications of the DCNN 198.

The body heatmap 200 is preferably generated using an UD criteria analysis leveraging the deep feature layer 206 of the DCNN 198. As described above, the deep feature layer 206 preferably is a last layer, or close to a last layer, of the output of the DCNN 198. The deep feature layer 206 preferably includes the deep features from the DCNN 198 of the classifications. Using the deep feature layer 206, the system 190 preferably is an image of a subject or portion thereof with a saliency overlay highlighting or color-coding skin abnormalities for use by clinicians when conducting referral decisions based on the UD criterial analysis. For example, subtle lesions are marked with blue, NSPL-A classified lesions are marked with green, NSPL-B classified lesions are marked with yellow, and SPL classified lesions are marked with red.

The body heatmap 200 preferably identifies each of the overlay portions that are color-marked according to the UD criteria analysis using the areas of interest identified by the trained image segmentation model 196. The DCNN 198 is then used on each cropped, rescaled single-lesion (e.g., skin abnormality) image for classification (e.g., class inference) and the areas of interest are overlaid with the original image of the subject.

The labeled lesion graphic 204 is preferably a blown-up portion of the body heatmap 200 showing a single lesion. Preferably the graphic 204 includes additional information that can assist a clinician in assessing whether to refer a patient for further testing or biopsy. The body heatmap 200 preferably is implemented in a user interface 108 so that various individual skin lesions may be selected and a blown-up version displayed as the graphic 204.

Referring now to FIG. 7, FIG. 7 is a functional block diagram of a skin abnormality detection system and method in accordance with a preferred embodiment of the present invention. In a preferred embodiment, the system 208 includes an imaging device 102, a datasets 210, a database 212, a trained image segmentation model 214, specialist trained AI models 216-224, and a user interface 226.

The imaging device 102 has been described extensively herein and will not be repeated for brevity purposes. The datasets 210 and the database 212 are largely similar to the datasets 192 and the database 194 in accordance with FIG. 6. However, the datasets 210, and therefore, the data stored on the database 212, may include a variety of additional or complementary datasets corresponding with skin conditions, eczema, hives, and contact dermatitis, autoimmune skin conditions, bacterial, viral, and fungal infections, burns, scars, and grafts, childhood skin conditions, skin cancers, and psychosomatic skin disorders, among others.

The trained image segmentation model 214 is also largely similar to the trained image segmentation model 196 and will not be repeated for brevity purposes. However, in a preferred embodiment, the trained image segmentation model 214 is a generalist image segmentation model that classifies skin conditions and/or abnormalities that are used to route the images to a specific specialist trained AI model (e.g., one or more of the specialist AI models 216-224). For example, the specialist trained AI models may correspond to the disciplines identified in Table 1 below. These disciplines each may include a common skin focus, as disclosed in Table 1. The disciplines and common skin focuses of Table 1 are non-limiting examples.

The model 214 preferably still includes the functions of the model 196 of FIG. 6 in conducting a search and identification of areas of interest that are cropped, stored, and retrieved for later use by a DCNN or other analytical tool or method.

TABLE 1
Disciplines and Common Skin Focuses

| Discipline | Common Skin Focus |
|---|---|
| Dermatology | Full spectrum of skin conditions |
| Dermatopathology | Biopsy and lab diagnosis |
| Immunology/allergy | Eczema, hives, contact dermatitis |
| Rheumatology | Autoimmune skin conditions |
| Infectious disease | Bacterial, viral, fungal infections |
| Burn/plastic surgery | Burns, scars, grafts |
| Pediatrics | Childhood skin conditions |
| Oncology | Skin cancers |
| Psychiatry | Psychosomatic skin disorders |
| General/internal medicine | Common first-line care |

Thus, each of the disciplines of Table 1 preferably include one or more datasets corresponding to the discipline's common skin focus. For example, the datasets 210 may include a dataset of images of eczema. The images of eczema may be captured with the imaging device 102 and/or be retrievable from public-access repositories. Other examples exist in connection with the listed disciplines and related common skin focus.

In addition, each of the specialist trained AI models 216-224 preferably corresponds to a discipline identified in Table 1. Thus, for example, a dermatology trained AI model 216 is trained on a full spectrum of skin conditions. The model 216 may therefore be identical to the DCNN 198 of FIG. 6. Each of the AI models 216-224 may be DCNNs. In another embodiment, the model 216 is a DCNN, but trained with different classifications and/or encompassing additional or varied skin conditions beyond those identified herein in connection with the classifications described in accordance with the DCNN 198. In another example, an eczema trained AI model 218 is trained on a dataset consisting of images of eczema. The model 218 may include classifications relating to a standardized eczema scoring system such as Eczema Area and Severity Index (EASI), Scoring Atopic Dermatitis (SCORAD), Patient-Oriented Eczema Measure (POEM), or customized scoring systems. In yet another example, a burn trained AI model 220 is trained on a dataset consisting of images of burns on patients' skin. The model 220 may include classifications relating to a standardized burn scoring system such as the Revised Baux score, Total Body Surface Area (TBSA) burned (expressed as a percentage), or customized scoring systems. While there are only five trained AI models 216-224 depicted in FIG. 7, the number is non-limiting and may consist of greater or lesser trained AI models 216-224.

Each of the trained AI models 216-224 may include heatmaps and/or montages similar to that described in connection with the body heatmap 200 and the lesion montage 202 depicted in FIG. 6. A person of ordinary skill in the art would understand that instead of lesions, a heatmap may highlight eczema, burns, or the like, and different criteria may be applied rather than UD criteria analysis, which is primarily used in dermatology for identifying potential melanoma. The heatmaps and/or montages may be displayed on the user interface 226 (e.g., user interface 108). The user interface 226 may be selectively toggled between different menus corresponding to the different disciplines. As described herein, the user interface 226 preferably includes historical images and/or data to be displayed alongside, or in combination, with currently captured images and/or data for use by the clinician, subject, or other user, and to draw conclusions therefrom.

PCPs often serve as the first point of contact for patients with skin lesions or other skin conditions, but they may lack the specialized training required for accurate diagnosis. The systems 192, 208, combined with AI-driven analysis, may assist PCPs with a powerful tool for more reliable and efficient SPL assessment. By enabling early and accurate triage, the systems 192, 208 may reduce the need for unnecessary referrals, thereby improving patient outcomes and optimizing healthcare resources. Given the projected shortage of PCPs in the coming years, the adoption of systems such as the systems 192, 208 may play a crucial role in addressing this gap. By empowering PCPs to manage a greater proportion of skin cancer screenings, the systems 192, 208 have the potential to significantly reduce diagnostic delays and enhance early detection efforts.

Referring now to FIG. 8, FIG. 8 is a flowchart illustrating a body change detection method 300 in accordance with a preferred embodiment of the present invention. For example, the method 300 utilizes the system 132 in connection with FIG. 3.

At Step 302, an image scan of a user is captured. As disclosed herein, the image scan is preferably a 360-degree image scan that comprises 360 individual images taken at each degree about the circumference of the user. The user is preferably an individual that is desiring clinical or other advice concerning their body, body change, and the like. It may also relate to a weight-loss clinic in which the clinician is measuring changes in the body of the user. While “image scan” is used in the description of this step, other images or a single image is within the scope of the present invention. At Step 304, datasets are obtained and stored. For example, the datasets 134 associated with the body part detection system 132 are disclosed herein and generally include images relating to parts of the body.

The datasets include both images or image scans of the user, as well as images or image scans of other users. In another embodiment, the datasets include public-access repositories and the like. At Step 306, the image scan and the datasets are stored in a database. The database is described in detail herein, and more particularly the database 144 in connection with FIG. 3. Alternatively, the datasets themselves are stored in a dataset database separate from the image scan. In other embodiments, the image scan is combined with the datasets to further augment the datasets for training AI models.

At Step 308, a detection and segmentation AI model is trained. The training of a detection and segmentation AI model is described in detail, and in particular, in accordance with the detection and segmentation model 146, the trained AI model 182, the trained image segmentation model 196, and the trained image segmentation model 214. The segmentation function of the AI model may be separately trained from the detection portion of the AI model. The detection and segmentation AI model may be trained for classifications corresponding to the datasets (e.g., body parts).

At Step 310, the image scan is classified using the trained detection and segmentation AI model. Classifying the image scan preferably includes inputting the image scan (e.g., preferably 360 images at the 360 degrees about the circumference of the user) and detecting body parts using the classifications of body parts that the AI was trained on, and outputting inferential data that includes a segmentation of the image by pixel.

At Step 312, segmented image data and metadata is generated. This may be the output of the trained detection and segmentation AI. It may also be a separately implemented algorithm or process that labels and tags the image segments with metadata so that segmented image data and metadata are generated. The segmented image data may also comprise a full body image of the user.

At Step 314, the segmented image data and metadata and historical image data and metadata of the user are compared. For example, the comparison is conducted by the body part analysis engine 148. As one example, the segmented image data is a full body image of the user that is compared with historical full body images of the user previously captured by the system 100 or other suitable system (e.g., system 132). Other examples include segmented image data representing only a portion of the user's body, such as an arm, leg, torso, nose, head, or the like. The segmented image data may also be a 3D image or video. As a person of ordinary skill in the art would recognize, the 360-degree imaging device 102 is capable of capturing 30 fps video that is capable of being reproduced as video or individual images.

At Step 316, an image and tags overlays are generated using the segmented image data. For example, the image and tags overlays are generated using the tagging engine 150 depicted in FIG. 3.

At Step 318, current and historical image data and tags overlays are displayed to a clinician.

Other functions such as identifying the user as a particular individual based on the segmented image data and metadata may be accomplished by the body change detection method 300 without departing from the scope of the present invention. The applications are non-limiting; while the method 300 is disclosed as being used in connection with weight-loss, clinical analysis, or the like, it may also be used in connection with law enforcement or other applications.

An SMT detection method is substantially similar to the body part detection method 300 disclosed herein. Functions such as identifying the user as a particular individual based on SMT data are within the scope of the present invention. The applications are non-limiting; while an SMT detection method is disclosed as being used in connection with tracking or identifying patients in a clinical setting, or the like, it may also be used in connection with law enforcement or other applications.

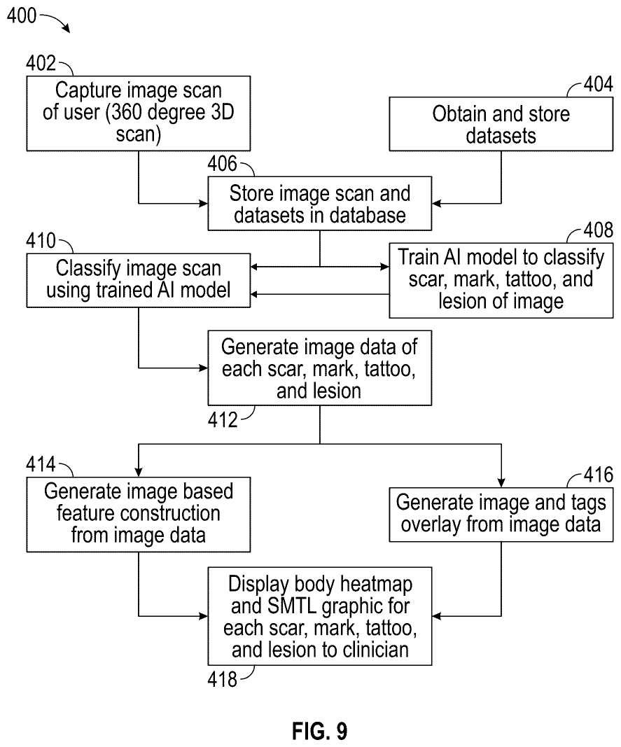

Referring now to FIG. 9, FIG. 9 is a flowchart illustrating a skin abnormality detection method 400 in accordance with a preferred embodiment of the present invention.

At Step 402, an image scan of a user is captured. At Step 404, datasets are obtained and stored. At Step 406, the image scan and the datasets are stored in a database. The image scan, datasets, and database are disclosed extensively herein, particularly in connection with the system 178 and the method 300, and are not repeated for brevity purposes.

At Step 408, an SMTL AI model is trained. Training the SMTL AI model is disclosed extensively herein, particularly in connection with the trained AI model 182 of FIG. 5.

At Step 410, the image scan is classified using the SMTL AI model. Classifying the image scan preferably includes inputting the image scan (e.g., preferably 360 images at the 360 degrees about the circumference of the user) and detecting scars, marks, tattoos, and lesions using the classifications of SMTL images that the AI was trained on, and outputting inferential data that includes a segmentation of the image by pixel.

At Step 412, image data and metadata is generated of each scar, mark, tattoo, and lesion. This may be the output of the trained AI model. It may also be a separately implemented algorithm or process that labels and tags the image segments with metadata so that segmented image data and metadata are generated. The segmented image data may also comprise a full body image of the user.

At Step 414, image-based feature construction is generated from the image data and metadata. The features preferably are those classified as scar, mark, tattoo, or lesion image segments. The construction of the features may include the segmented images/image data and metadata, along with a comparison of the scar, mark, tattoo, or lesion image segments against images or image segments in the database.

At Step 416, image and tags overlays are generated from the image data and metadata. The SMTL image segments and overlays preferably relate to the SMTL data classified by the trained AI model.

At Step 418, a body heatmap and an SMTL graphic is displayed to a clinician for each scar, mark, tattoo, and lesion. The body heatmap preferably identifies lesion data, similar to that disclosed by the body heatmap 184 or the body heatmap 200. The SMTL graphic preferably identifies scar, mark, tattoo, or lesions overlayed by tag overlays, similar to that disclosed as the lesion graphic 188 of FIG. 5, or the lesion montage 202 of FIG. 6. While the flowchart specifies a “clinician” to which the body heatmap and SMTL graphic is displayed, the subject user or other users may view the display, preferably using the user interface 108, to view the information and draw conclusions therefrom.

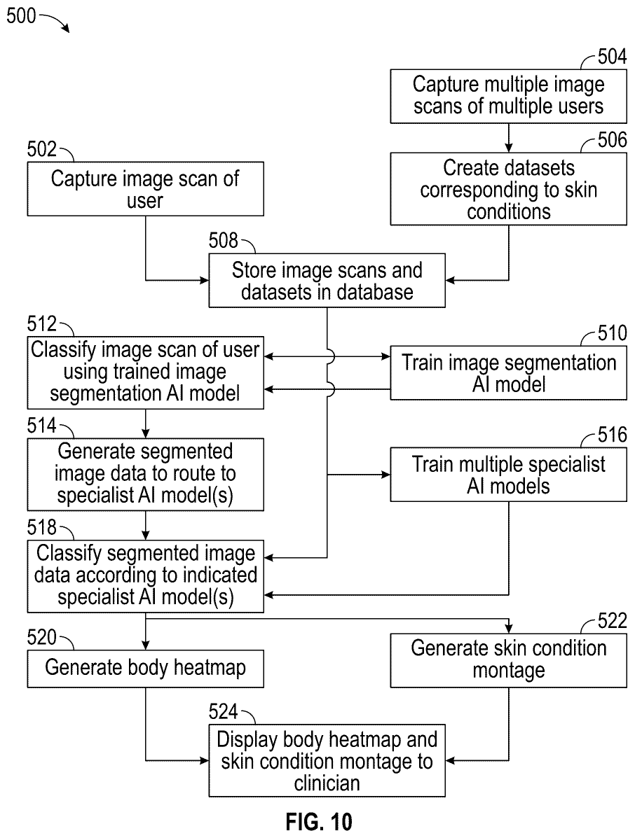

Referring now to FIG. 10, FIG. 10 is a flowchart illustrating a skin condition detection method 500 in accordance with a preferred embodiment of the present invention.

At Step 502, an image scan of a user is captured. At Step 504, multiple image scans of multiple users are captured. At Step 506, datasets are obtained corresponding to skin conditions. At Step 508, the image scans and the datasets are stored in a database. The image scans, datasets, and database are disclosed extensively herein, particularly in connection with the system 190, the system 208 and the method 300, and are not repeated for brevity purposes. Here, however, the multiple image scans are preferably 360-degree image scans that comprise 360 separate images, as disclosed herein. The use of such image scans as both the input data for the AI models and the training data used to train the AI models may provide significant advantages in accuracy and lead to simplification of neural networks so that more efficient analysis may be accomplished. Other combinations of datasets exist that include public-access repositories, and the like.

At Step 510, an image segmentation AI model is trained. Training the image segmentation AI model is disclosed extensively herein, particularly in connection with the trained AI models 196, 214 of FIGS. 6-7.

At Step 512, the image scan is classified using the trained image segmentation AI model. Classifying the image scan preferably includes inputting the image scan (e.g., preferably 360 images at the 360 degrees about the circumference of the user) and detecting skin conditions using the classifications of skin conditions that the AI was trained on, and outputting inferential data that includes a segmentation of the image by pixel.

At Step 514, segmented image data and metadata is generated to route to one or more specialist AI models based on the skin conditions. The system 208 of FIG. 7 is suitable to generate the segmented image data and metadata for a variety of skin conditions corresponding with the classifications of skin conditions on which the AI model was trained. Thus, the method corresponding to the system 208 of FIG. 7 may be configured to route the segmented image data and metadata to one or more of the specialist AI models 216-224, each corresponding to different disciplines relating to the skin condition classifications. The system 198 of FIG. 6 includes only segmented image data and metadata corresponding to skin abnormalities such as skin lesions. Thus, the method corresponding to the system 198 of FIG. 6 may be configured to route the segmented image data and metadata to the specialist AI model 216, i.e., the dermatology trained AI model 216, corresponding to skin lesion classifications.

At Step 516, multiple specialist AI models are trained that correspond to clinical disciplines associated with the skin conditions. The training of the specialist AI models 216-224 are disclosed extensively herein and are not repeated for brevity purposes. However, the separate specialist AI models 216-224 are configured to provide a quicker and more efficient system so that the particular AI models are not trained on classifications irrelevant to those skin conditions. For example, a UD criteria analysis may not be appropriate for eczema skin conditions, burn skin conditions, and the like, but are suitable for use in connection with skin lesion conditions.

At Step 518, the segmented image data is classified according to the indicated specialist AI model(s). Classifying the segmented image data preferably includes inputting the segmented image data extracted, cropped, and stored from the image scan (e.g., preferably 360 images at the 360 degrees about the circumference of the user) and detecting skin conditions using the classifications of skin condition images that the AI was trained on, and outputting inferential data that includes classifications of skin conditions associated with the particular discipline for which the specialist AI model is suitable to classify.

At Step 520, a body heatmap is generated from the data output by the indicated specialist AI model. For example, the body heatmap preferably identifies lesion data, similar to that disclosed by the body heatmap 184 or the body heatmap 200.

At Step 522, a skin condition montage is generated from the data output by the indicated specialist AI model. For example, the skin condition montage preferably discloses skin condition data, similar to that disclosed by the lesion graphic 188 of FIG. 5, the lesion montage 202 of FIG. 6, or through the user interface 108, as shown in connection with FIGS. 6-7. A person of ordinary skill in the art will appreciate that the skin condition montage is specific to the particular output of the indicated specialist AI model.

At Step 524, the body heatmap and the skin condition montage are displayed to a clinician. While the flowchart specifies a “clinician” to which the body heatmap and the skin condition montage is displayed, the subject user or other users may view the display, preferably using the user interface 108, to view the information and draw conclusions therefrom.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A method of detecting skin abnormalities, the method comprising the steps of
capturing a first image scan,
identifying areas of interest of the first image scan using a trained image segmentation model, wherein the trained image segmentation model is trained using a first plurality of image scans,
classifying the areas of interest in accordance with classifications of a trained deep neural network trained with a second plurality of image scans, wherein the classifications include at least one of suspicious pigmented lesions (SPLs), medium-priority nonsuspicious pigmented lesions (NSPLs), and low-priority NSPLs,
extracting a deep feature layer of the trained neural network for each of the areas of interest,
calculating an ugly duckling metric for each of the areas of interest by comparing a geometric distance of the deep feature layer with an average of the deep feature layer for the areas of interest,
displaying the ugly duckling metric associated with each of the areas of interest in accordance with the image scan, and
displaying ranked areas of interest identified in the first image scan ranked in accordance with the classifications.

2. The method of claim 1 wherein each of the first image scan and the first and second pluralities of image scans comprise a 360-degree view of at least a portion of a body part.

3. The method of claim 1 further comprising storing the first image scan in a database, wherein the database comprises a dataset of images of body parts including the first image scan.

4. The method of claim 3 wherein the dataset of images includes the first and second pluralities of image scans.

5. The method of claim 1 wherein the areas of interest include at least one of lesions, burns, moles, freckles, tan lines, wrinkles, cuts, bruises, scars, marks, tattoos, bumps, and lumps.

6. The method of claim 1 wherein the first image scan comprises 360 individual images with consistent lighting, position, and backdrop.

7. The method of claim 1 wherein the first image scan is captured with a 360-degree imaging device that comprises a rotating unit that includes an imaging camera, wherein the rotating unit is rotatable between a home position and a finish position about a rotation axis such that the imaging camera is configured to capture the first scan, an alignment camera configured to capture a first alignment image of a subject positioned generally co-axially with the rotation axis, and a first display on which the first alignment image is displayed.

8. The method of claim 1 wherein the first image scan is captured with a 360-degree imaging device that comprises a rotating unit that includes an imaging camera, wherein the rotating unit is rotatable about a rotation axis, wherein the rotating unit includes a first portion and a second portion that are rotatable about the rotation axis, wherein the first portion and the second portion each have a proximal end through which the rotation axis extends and a distal end that is positioned away from the rotation axis, wherein the imaging camera is associated with the distal end of the first portion and a backdrop is associated with the distal end of the second portion, wherein the first portion and the second portion are rotatable from a home position where the distal ends of the first portion and the second portion are approximately 180° from one another with respect to the rotation axis, and wherein the first portion and the second portion are rotatable together between the home position and a finish position such that the imaging camera can capture the first scan of a subject positioned generally co-axially with the rotation axis.

9. The method of claim 1 wherein the first image scan is displayed with an overlay indicating the ugly duckling metric.

10. The method of claim 1 wherein the first image scan is displayed with an overlay indicating the ranked areas of interest.

11. The method of claim 1 wherein the ugly duckling metric is calculated using a portion of the first image scan.

12. The method of claim 1 wherein the ugly duckling metric is displayed as a saliency map.

13. The method of claim 1 wherein the ugly duckling metric is displayed as a body heatmap.

14. The method of claim 1 wherein the ranked areas of interest are displayed as a montage.

15. A method of detecting skin conditions, the method comprising the steps of
capturing a first image scan,
identifying areas of interest of the first image scan using a trained custom image segmentation model, wherein the trained custom image segmentation model is trained using a first plurality of image scans,
classifying each of the areas of interest to correspond to skin conditions, wherein the skin conditions include at least one of eczema, hives, contact dermatitis, autoimmune skin conditions, bacterial infections, viral infections, fungal infections, burns, scars, grafts, childhood skin conditions, skin cancers, and psychosomatic skin disorders, routing each of the areas of interest to at least one trained specialist model, the trained specialist model corresponding to one or more of the skin conditions, wherein the at least one trained specialist model is a deep neural network trained using a second plurality of image scans,
classifying the areas of interest using the at least one trained specialist model,
extracting a deep feature layer of the at least one trained specialist model for each of the areas of interest,
calculating a skin condition metric for each of the areas of interest using the deep feature layer, the skin condition metric corresponding to a standardized scoring system for evaluating at least one of the skin conditions,
displaying the skin condition metric associated with each of the areas of interest in accordance with the first image scan,
displaying ranked areas of interest identified in the first image scan ranked in accordance with the classifications.

16. The method of claim 15 wherein each of the first image scan and the first and second pluralities of image scans comprise a 360-degree view of at least a portion of a body part.

17. A skin condition detection system comprising
an imaging device configured to capture a first image scan of a first user,
a plurality of datasets that include a plurality of image scans of multiple users,
a database configured to store the first image scan and the plurality of image scans,
a trained image segmentation model, wherein the trained image segmentation model is trained using the plurality of image scans stored on the database, and is configured to identify areas of interest corresponding to classifications of skin conditions and to output segmented image data for each of the areas of interest,
at least one trained specialist model configured to receive the segmented image data and to classify the segmented image data for each of the areas of interest for at least one of the skin conditions, and
a display configured to display a skin condition metric based on a standardized scoring system for evaluating at least one of the skin conditions for each of the areas of interest, and ranked areas of interest identified in the first image scan ranked in accordance with the classified segmented image data.

18. The system of claim 17 wherein the first image scan comprises a 360-degree view of at least a portion of a body part.

19. The system of claim 17 wherein the plurality of image scans of multiple users comprises at least one 360-degree view of at least a portion of a body part.

20. The system of claim 17 wherein the plurality of image scans of multiple users include images of at least one skin condition.

21. The system of claim 17 wherein the at least one trained specialist model is a deep convolutional neural network.

22. The system of claim 17 wherein the skin condition metric is calculated using a deep feature layer of the deep convolutional neural network in accordance with the standardized scoring system.

23. The system of claim 17 wherein the at least one trained specialist model corresponds to one or more of the skin conditions.

24. The system of claim 17 wherein the skin conditions include at least one of eczema, hives, contact dermatitis, autoimmune skin conditions, bacterial infections, viral infections, fungal infections, burns, scars, grafts, childhood skin conditions, skin cancers, and psychosomatic skin disorders.
